(19) United States
(12) Patent Application Publication
(10) Pub. No.: US 2012/0136258 A1
(43) Pub. Date: May 31, 2012

(54) Retinal Flow Cytometry

(75) Inventors: Charles P. Lin, Arlington, MA (US); Clemens Alt, Watertown, MA (US); Israel Veilleux, Saint-Nicolas (CA); Daniel Cote, Des Charmes (CA)

(73) Assignee: The General Hospital Corporation D/B/A Massachusetts General Hospital, Boston, MA (US)

(21) Appl. No.: 13/357,939

(22) Filed: Jan. 25, 2012

Related U.S. Application Data
(62) Division of application No. 12/114,413, filed on May 2, 2008.
(60) Provisional application No. 60/927,562, filed on May 4, 2007.
(60) Provisional application No. 60/927,853, filed on May 4, 2007.
(60) Provisional application No. 60/988,525, filed on Nov. 16, 2007.

(51) Int. Cl. A61B 6/00

(52) U.S. Cl. 600/476

(57) ABSTRACT

The present invention provides methods and devices for performing flow cytometry. In one embodiment, blood circulating through one or more retinal blood vessels of a subject is illuminated in-vivo so as to excite a plurality of fluorescent-labeled cells contained in the blood. The fluorescence radiation emitted by the excited cells is then detected and analyzed to count the cells from which fluorescence is detected.

### RELATED APPLICATIONS

This application claims priority to a provisional application entitled, “Method and System for Performing Flow Cytometry In Vivo,” filed on May 4, 2007 and having a Ser. No. 60/927,562, provisional application entitled “Method and System for Performing Flow Cytometry In Vivo,” filed on May 4, 2007 and having a Ser. No. 60/927,853; and provisional application entitled, “Retinal Flow Cytometry,” filed on Nov. 16, 2007 and having a Ser. No. 60/988,525. These provisional applications are herein incorporated by reference in their entirety.

### GOVERNMENT SUPPORT

This invention was made with government support under NIH/BRP contract number EY 014106. The U.S. government has certain rights in this invention.

### FIELD

The present application relates to methods and devices for performing flow cytometry, and more particularly, it is directed to such methods and devices for conducting real-time in vivo quantification of the flow characteristics of a subject's circulating cells through the retinal blood vessels.

### BACKGROUND

Current methods for detecting and quantifying various types of cells circulating within a subject's blood stream typically involve extraction of blood from the subject (a patient or an animal) followed by labeling and ex vivo detection. For example, in standard flow cytometry, specific cell populations in a blood sample, drawn from a subject and fluorescently labeled, are passed in single file through a flow stream to be interrogated by a light source (usually a laser). Fluorescence and light scattering signals emitted, or remitted, by the cells in response to the light source can be employed to determine the types and the number of the cells. In another ex vivo conventional technique, known as hemocytometry, cells are counted against a grid while being viewed with a microscope to determine the types of the cells and their numbers.

Such ex vivo techniques, however, suffer from a number of shortcomings. For example, each measurement provides only a single time sample. Consequently, it is difficult to use these techniques to obtain a valid temporal population profile for a cell type of interest that varies unpredictably or rapidly with time. Further, these techniques can suffer from a significant time delay between sample collection and analysis, leading to potential measurement inaccuracies.

Some in vivo techniques for detection of static and circulating fluorescently labeled cells are also known. However, these techniques typically show difficulty, or simply fail, in tracking cells flowing at a high velocity, especially in the arterial circulation, even when they capture images at video rates. In addition, employing these techniques for extracting quantitative information about the number and flow characteristics of a specific cell population can be tedious.

Hence, there is a need for enhanced methods and apparatus for performing in vivo flow cytometry.

### SUMMARY

In one aspect, the present invention provides a method for performing flow cytometry by illuminating in-vivo blood circulating through one or more retinal blood vessels of a subject so as to excite a plurality of fluorescent-labeled cells contained in the blood. The fluorescence radiation emitted by the excited cells can be detected and analyzed to count the cells from which fluorescence radiation is detected. Such a cell count can be used to obtain information about one or more cell types of interest. By way of example, the information can include a volume density of a selected cell type circulating through the subject. The term “illuminating in vivo” refers to illuminating the blood in a live subject (human or animal) while the blood is circulating through the subject.

In a related aspect, the illuminating step can include scanning a light beam over the retina in a predefined pattern, such as a circular pattern. In some cases, the light can be scanned over the retina in the circular pattern at a rate such that each of a plurality of cells is intercepted at least once. By way of example, the light can be scanned at a rate in the range of about 100 Hz to 100 kHz. In one embodiment, the light can be scanned at a rate of greater than about 1000 Hz. In one exemplary embodiment, the pattern can be in the form of a plurality of disjointed segments, each of which corresponds to illuminating a retinal vessel.

In another aspect, the fluorescence detection can be performed confocally relative to the excitation. Such confocality allows detecting fluorescence from a selected excitation volume while minimizing interference from radiation emanating from regions outside that excitation volume.

In another aspect, the invention provides a method for performing flow cytometry by introducing a fluorescence marker into a subject's circulating blood so as to label a plurality of cells with the marker, and illuminating a portion of the subject's retina in a selected pattern so as to excite fluorescent-labeled cells circulating through a plurality of retinal blood vessels. The fluorescence radiation emitted by the excited labeled cells can be detected and analyzed. While in some embodiments, such detection can be performed confocally relative to excitation, in other embodiments confocal detection is not utilized.

In a related aspect, the circulating cells can be labeled by introducing the probe molecules into the subject's circulatory system. For example, the probe molecules can include, e.g., a fluorescent marker that can couple to a membrane protein of the plurality of cells. By way of example, a fluorescent probe can be a fluorescently labeled antibody capable of binding to a surface antigen of a cell type of interest. The fluoresecence markers (probes) are not limited to antibodies. In fact, the fluorescence marker can be any suitable marker, e.g., membrane-embedded, surface-bound, endocytosed, etc.

A variety of different cell types can be labeled with such fluorescent probes. Some examples of such cell types include, without limitation, leukocytes (lymphocytes, monocytes, granulocytes), tumor cells, and stem cells.

In another aspect, the fluorescence radiation can be analyzed to derive information regarding the plurality of cells. For example, the derived information can provide a cell count of the plurality of cells relative to a corresponding count measured previously. In some cases, such a relative cell count can be indicative of progress of a disease or of a treatment protocol applied to the subject. In some embodiments, the derived information can provide an absolute cell count of the plurality of cells. The absolute cell count can be indicative of any of presence of a disease and/or progress of a treatment protocol.

In another aspect, the invention provides a method for performing flow cytometry by labeling one or more cells of a selected type of a subject with one or more fluorescent probe molecules while the cells circulate in the subject. An excitation radiation beam can be scanned over a selected area of the subject to excite the one or more fluorescent probe molecules. Fluorescence radiation emitted by the one or more fluorescent probe molecules in response to the excitation radiation can be detected.

In a related aspect, the detected fluorescence radiation can be analyzed so as to derive information regarding the circulating cells of the selected type. In some cases, analyzing the fluorescence radiation can include determining whether a signal-to-noise ratio (SNR) of a detected fluorescence signal exceeds a pre-defined threshold. If the intensity exceeds such a threshold, the fluorescence signal can be identified as emanating from an excited cell.

In a further aspect, a rate of the scan is such that one or more cells flowing through a vessel are illuminated multiple times as the beam is scanned over the retina. In some embodiments, a cell count can be registered (identified) when a pre-defined number of detected fluorescence signals collected from a vessel over a time period shorter than a time interval required for passage of a labeled cell through an illuminated portion of the vessel exhibit an intensity exceeding the threshold. The derived information can provide a cell count of the plurality of cells relative to a corresponding count measured previously. As noted above, such a relative cell count can be indicative, e.g., of progress of a treatment protocol applied to the subject. In other cases, the derived information can provide an absolute cell count of the plurality of cells. The absolute cell count can be indicative of any of presence of a disease and/or progress of a treatment protocol

In another aspect, the invention provides a method for performing flow cytometry by directing a scanning radiation beam to a subject's retina. The radiation can have one or more wavelengths capable of exciting one or more fluorescent-labeled cells circulating through a plurality of retinal blood vessels. The radiation beam can be selectively activated as the beam traverses a retinal blood vessel to excite one or more fluorescent-labeled cells traveling through that vessel. Fluorescence radiation emitted by the excited fluorescent-labeled cells can be detected and analyzed. In some embodiments, the method can also include deactivating the radiation as the scanned beam illuminating a retinal blood vessel leaves that vessel to enter a retinal region substantially free of blood vessels.

In another aspect, the invention provides a method for performing flow cytometry by directing a scanned radiation beam to a subject's retina. An intensity of the beam can be modulated so as to selectively illuminate a plurality of retinal vessels in order to excite one or more fluorescent-labeled cells circulating through the vessels. Fluorescence radiation emitted by the excited cells can be detected and analyzed to count the cells from which fluorescence radiation is detected and to derive information about one or more cell types of interest.

In another aspect, the invention provides a method for performing flow cytometry by selectively illuminating a plurality of vessels in a temporal sequence so as to excite one or more fluorescent labeled cells circulating through the vessel. Fluorescence radiation emitted by the one or more fluorescent labeled cells can be detected and analyzed to count the cells from which fluorescence is detected and to derive information about one or more cell types of interest.

In another aspect, the invention provides a system for performing flow cytometry that includes a radiation source for generating radiation having one or more wavelength components capable of exciting a fluorescent marker suitable for binding to at least one type of cells circulating in a subject. A scanning mechanism can be optically coupled to the source and adapted to cause a two-dimensional scan of the radiation. A modulation mechanism can be adapted to modulate the intensity of the radiation, and an optical system can direct the scanned radiation to a tissue portion of the subject.

In another aspect, the invention provides a system for performing flow cytometry that includes a radiation source for generating radiation having one or more wavelength components capable of exciting a fluorescent marker suitable for binding to at least one type of cells circulating in a subject. A scanning mechanism can be optically coupled to the source and adapted to cause a two-dimensional scan of the radiation, and an optical system can be used for directing the scanned radiation to a tissue portion of the subject.

In a further aspect, the optical system is adapted to image the scanned radiation onto a focal plane in which a tissue portion can be exposed to the radiation. The system for performing flow cytometry can also include a detector for detecting fluorescence radiation emitted by the one or more fluorescent probe molecules. In some cases, the detector can be configured for confocal detection of the fluorescence radiation. The system can also further include an analysis module coupled to the detector for analyzing the fluorescence radiation so as to derive information regarding the circulating cells.

### BRIEF DESCRIPTION OF THE DRAWINGS

The invention will be more fully understood from the following detailed description taken in conjunction with the accompanying drawings, in which:

FIG. 1 is a flow chart depicting various steps in one embodiment of a method according to the teachings of the invention for performing retinal flow cytometry using a scanned beam of radiation;

FIG. 2 is a schematic illustration of a radiation beam being scanned in a selected pattern over the retina so as to illuminate a plurality of retinal blood vessels.

FIG. 3A is an exemplary illustration of a confocal fluorescence image of retinal blood vessels visualized with a fluorescent dye that homogenously distributes within the blood;

FIG. 3B is an exemplary illustration of mapping the circular scans of FIG. 3A into horizontal lines, which results in the display of the blood vessels as straight vertical features;

FIG. 4 is a schematic that illustrates a system according to one embodiment of the invention for performing retinal flow cytometry;

FIG. 5 is a schematic illustration of one exemplary method for analyzing fluorescence signals obtained from the excited labeled cells in retinal blood vessels to determine the presence of a labeled cell;

FIG. 6 is a schematic illustration of a comparison of graphs of fluorescence signals of retinal blood vessels to determine the presence of a labeled cell;

FIG. 7 is a schematic illustration of another exemplary method for analyzing fluorescence signals obtained from the excited labeled cells in retinal blood vessels to determine the presence of a labeled cell;

FIG. 8 is a flow chart depicting various steps in another embodiment of a method according to the teachings of the invention for performing retinal flow cytometry using a modulated scanned beam of radiation;

FIG. 9A is an exemplary illustration of a fluorescence image in retinal blood vessels visualized with cells labeled with fluorescent probe molecules using a modulated scanned beam of radiation;

FIG. 9B is an illustration of a graph showing acousto-optic modulator (AOM) command voltage versus time to control the modulated scanned beam of radiation as it scans the retinal blood vessels shown in FIG. 9A;

FIG. 10 is a schematic that illustrates a system according to another embodiment of the invention for performing retinal flow cytometry, which can be utilized to create the pattern in FIGS. 9A and 9B;

FIG. 11A schematically depicts another embodiment of an apparatus according to the invention for performing flow cytometry that utilizes a mask for projecting slit-shaped radiation beams onto a plurality of retinal blood vessels;

FIG. 11B is a schematic top view of the mask utilized in the apparatus of FIG. 11A;

FIG. 11C schematically shows the exposure of a plurality of retinal blood vessels to radiation passing through the mask shown in FIG. 11B;

FIG. 11D schematically shows another mask, projecting a stationary ring onto the retina, suitable for use in the apparatus of FIG. 11A;

FIG. 12A schematically shows an apparatus that creates a stationary ring on the retina by utilizing the donut-mode of an optical waveguide, according to another embodiment for performing flow cytometry; and

FIG. 12B schematically shows an apparatus that creates a stationary ring on the retina by coupling the excitation light into the cladding of an optical wave guide according to another embodiment for performing flow cytometry,

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

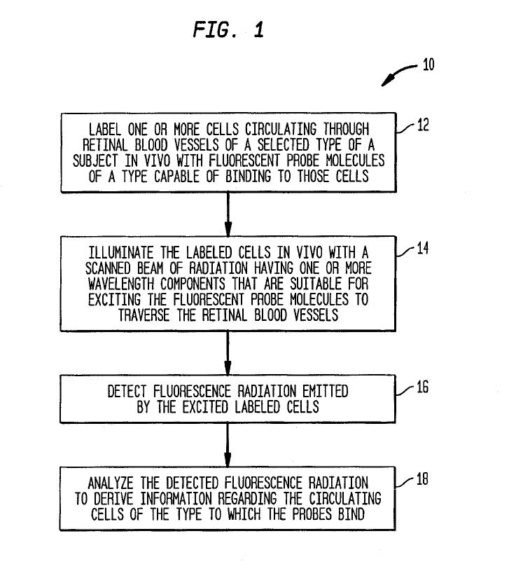

With reference to a flowchart 10 of FIG. 1, in one exemplary embodiment of a method according to the teachings of the invention for performing retinal flow cytometry, in an initial step 12, one or more cells of a selected type of a subject, e.g., a patient, are labeled in vivo, that is while circulating through the live subject, with fluorescent probe molecules of a type capable of binding to those cells. In other words, the cells are labeled while circulating through the subject, i.e., without extraction, ex-vivo labeling and re-introduction of the cells back into the subject. For example, the probe molecules can be injected into the subject's circulatory system to bind to these cells, which also circulate through the subject. The labeled cells then circulate through the subject's vascular system including retinal blood vessels. As discussed further below, it has been discovered that information regarding such circulating cells can be gleaned by illuminating one or more retinal blood vessels with radiation having one or more wavelengths that are suitable for exciting the fluorescent probes.

The probe molecules can, for example, couple to one or more surface proteins, e.g., membrane proteins, of the selected cells. In some embodiments, a fluorescent probe molecule can be a fluorescent-labeled anti-body that is capable of binding to a surface antigen of a cell type of interest. Such cell types can include, without limitation, leukocytes, tumor cells, and stem cells. Some examples of suitable antibodies include, without limitation, anti-CD4 for lymphocytes, and PSMA for prostate cancer cells.

Referring again to the flowchart 10, in step 14 one or more retinal blood vessels of the subject are illuminated in vivo, i.e., in the live subject, with radiation having one or more wavelength components that are suitable for exciting the fluorescent probes. In general, the probes are chosen such that they can be activated by radiation that can substantially penetrate through the subject's tissue and blood to reach them. In some embodiments, radiation suitable for activating the probes can have wavelength components in the infrared range of the electromagnetic spectrum. For example, radiation with wavelengths in a range of about 400 nm to about 1000 nm, and more preferably in a range of about 400 nm to about 800 nm, can be employed for exciting the probes. Although many different radiation sources can be utilized in the practice of the invention, in many embodiments, a laser source, such as, a He—Ne laser, generates radiation suitable for activating the probes. Further, in many embodiments of the invention, such as the embodiments discussed below, the radiation source generates a beam that is focused, e.g., by a series of lenses, onto a selected portion of a vessel of the subject.

By way of example, in some embodiments, a radiation beam is scanned in a selected pattern over the retina so as to illuminate a plurality of retinal blood vessels. For example, as shown schematically in FIG. 2, a radiation beam can be focused on the retina 20 to provide a radiation spot 21. The focused radiation spot 21 can be scanned along a circular path (shown by dashed lines) to illuminate successively a plurality of retinal blood vessels 22. In many cases, the rate at which the radiation spot 21 is scanned over the retina 20 is selected such that each of the vessels 22 is illuminated multiple times during the time it takes for a labeled cell to travel through that vessel 22 over a distance corresponding to a diameter of the radiation spot 21. In other words, in such cases, a labeled cell can be excited multiple times during its passage through a retinal vessel 22 illuminated by the scanning radiation spot 21. In other cases, the rate at which the radiation spot 21 is scanned over the retina 21 is selected such each cell is intercepted only once. A person skilled in the art will appreciate that the rate at which the radiation spot 21 scans the retina 21 can be chosen to intercept each cell any number of times, as long as each cell is intercepted at least once. It should be understood that the scan rate is dependent on the size of the cells to be illuminated and the blood flow velocity.

In some embodiments, the diameter of the illumination spot 21 over the retina 20 can be, e.g., in a range of about 0.3 to about 30 μm. Further, the power of the illuminating radiation on the retinal surface can be adjusted to provide a good fluorescence signal (the power can be typically in a range of about 0.1 to about 1 mW), where the maximum power is limited by ANSI standard. Although the illumination spot 21 is shown herein as having a circular cross-section, in other cases it can have other cross-sectional shapes, such as elliptical.

Referring again to the flow chart 10 of FIG. 1, following excitation, the excited labeled cells, and more particularly their attached fluorescent probe molecules, emit fluorescence radiation, which is typically red-shifted (i.e., it has a higher wavelength) relative to the excitation radiation. In step 16, this fluorescence radiation is detected. In one exemplary embodiment, the fluorescence radiation is confocally detected. The term “confocal detection” is known in the art, and to the extent that any further explanation is required, it refers to detecting the fluorescence photons in a plane that is conjugate to a plane of the excitation radiation that is focused onto a selected portion of a subject's circulatory system, e.g., a retinal vessel, to excite the probe molecules flowing therethrough.

In step 18, the detected fluorescence can be analyzed so as to derive information regarding the circulating cells of the type to which the probes bind. Such information can include, without limitation, the concentration of such cells in the subject's circulatory system, their average flow velocity, size and circulation lifetime. For example, in some embodiments, the fluorescence radiation can be analyzed to obtain a cell count of a particular cell type relative to a previously-measured cell count (e.g., by utilizing relative number of fluorescent peaks counted in a selected time interval). By way of example, such a relative cell count measurement can provide a medical practitioner with information regarding presence and/or progression of a disease and/or efficacy of a previously applied treatment. For example, the above method of invention can be utilized to derive a relative cell count of tumor cells of a particular type circulating through a patient's circulatory system, thereby allowing assessment of the effectiveness of a treatment protocol.

In some embodiments, the analysis of the fluorescence signal obtained from the excited labeled circulating cells include determining the presence of a labeled cell when the fluorescence signal is detected a predefined number of times within a region of interest covering a blood vessel in the retina. If the fluorescence signal is detected enough times, the signal is determined to represent a labeled cell traveling through that retinal blood vessel. In another embodiment, the analysis includes determining the presence of a cell by the pixel area of the fluorescence signal in a flow cytometer frame. If a fluorescence signal spans a number of horizontal pixels that indicate a width of a cell and if the same fluorescence signal also spans a number of vertical pixels that indicate that the fluorescence has been detected for a predefined number of times, then the signal is identified as arising from a cell.

In some embodiments, the detected fluorescence can be employed to determine an absolute cell count of the cell type of interest. The number of target cells of interest in a given probe volume of blood, at a given time, flowing through a vessel can be given by the following relation:

n=[C]*A*v*t

where [C] denotes the concentration of cells to be analyzed (e.g., number of cells/ml), A denotes the cross-sectional area of the vessel, v is an average flow velocity of blood through the vessel, and t is the sampling time. The product A*v*t denotes the probe volume. Parameter n is the measured cell number for a given measurement period t. Therefore, if A and v are known, then [C] can be determined. In many embodiments, vessel diameters in a range of about 10 to about 100 microns are employed for cell counting. Larger vessel can also be employed, e.g., for detecting tumor cells.

In an alternative embodiment, the labeling of the cells of interest with fluorescent probes is performed ex vivo, that is, after extraction of the cells from a subject. The labeled cells are then re-introduced into the subject's circulatory system, and are irradiated so as to excite the probes. The fluorescence radiation emitted by the excited probes is detected and analyzed to derive the desired cytometric information. Alternatively, fluorescent proteins can be expressed in a selected cell type of a subject, for example, by employing reporter genes (e.g., GFP).

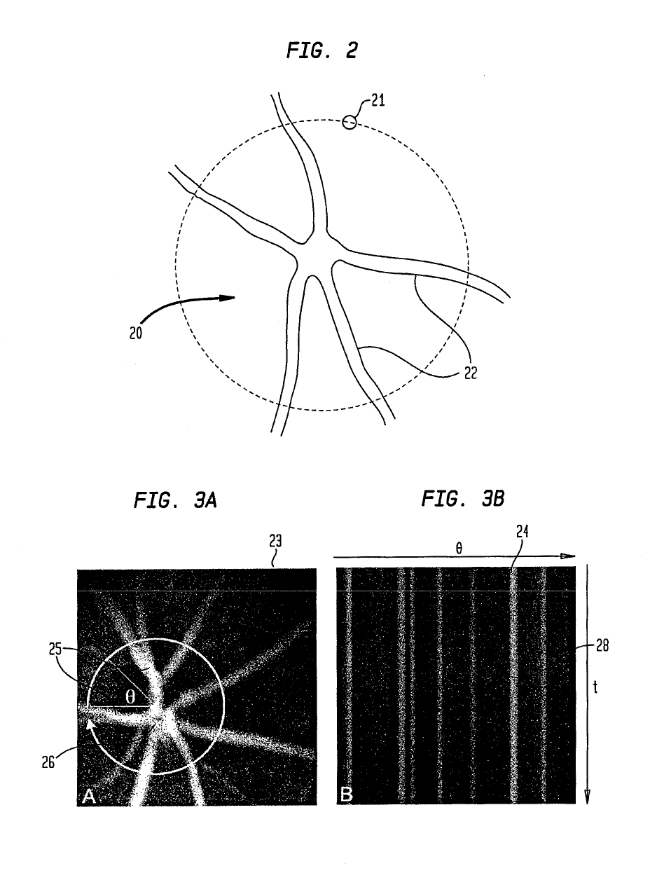

By way of illustration, FIG. 3A presents an exemplary confocal fluorescence image of retinal blood vessels 25 visualized with a fluorescent dye that distributes homogenously within the blood. A variety of different dyes can be used, including Evans blue. To obtain the image 23, a scanned beam of radiation was used to illuminate a plurality of retinal blood vessels 25 that diverge outwardly from the optic nerve heard 26. The circular scans shown in image 23 can be mapped to straight horizontal lines 28, as shown in image 24. Each vertical feature 28 shown in image 24 corresponds to a single retinal blood vessel 25, as shown in FIG. 3B.

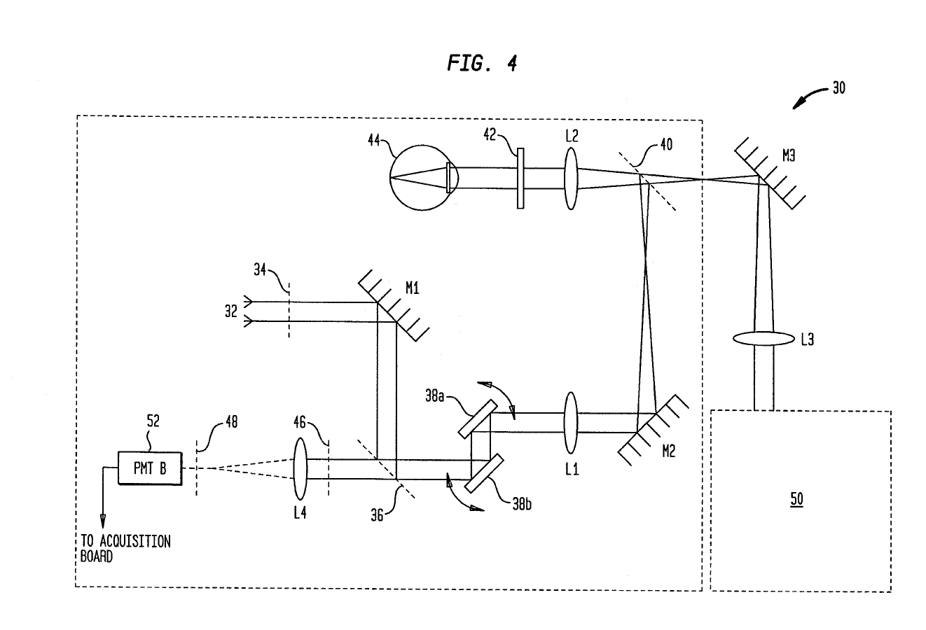

FIG. 4 schematically illustrates a system 30 according to one exemplary embodiment of the invention for performing retinal flow cytometry in accordance with the teachings of the invention, for example, a system by which the above described method of retinal flow cytometry can be practiced. The exemplary system 30 includes a radiation source 32 for generating a beam of photons suitable for exciting probe molecules previously administered to a subject under examination. In the illustrated embodiment, the radiation source 32 is a He—Ne laser that generates a continuous-wave (CW) lasing radiation at a wavelength of 633 nm. Without any limitation, in the illustrated embodiment, the He—Ne laser generates a laser beam having a substantially circular cross-section in a plane perpendicular to the propagation direction and a substantially Gaussian intensity profile in that plane. Those having ordinary skill in the art will appreciate the radiation beams having different cross-sectional shapes and/or cross-sectional intensity profiles can also be utilized. Moreover, a person having ordinary skill in the art will appreciate that the radiation source 32 can be other than a He—Ne laser so as long it provides radiation suitable for exiting the labeled cells. Other radiation sources can include (depending on the fluorescent molecule used to label the cells), but are not limited to, gas, diode and solid-state lasers ranging from the ultra-violet to the infra-red, at exemplary wavelengths of about 266, 375, 470, 490, 514, 532, 561, 750, 830 nm.

The radiation generated by the He—Ne laser passes through a neutral density filter 34 (NDF) that can adjust the radiation intensity to a desired level. Typically, the laser power is adjusted to yield a power on the cornea that is less than about 1 mW. A mirror M1 directs the radiation received from the source to a beam splitter or dichroic filter 36, which in turn transmits the radiation to a pair of scanning mirrors 38a and 38b that are rotatable about two mutually orthogonal axis. Each scanning mirror swivels about its respective rotational axis in a periodic fashion such that the two mirrors cooperatively scan the beam in a given pattern, e.g., circular. In this embodiment, the oscillation rates of the two mirrors are substantially equal to cause the beam to scan along a circular path. By way of example, the oscillation rate can be in a range of about 0.1 to 100 kHz, and in some cases in a range of about 1 kHz to about 10 kHz. A person skilled in the art will appreciate, however, that the minimum oscillation rate that is required to detect each cell at least once is determined by the size and velocity of cells flowing within the blood stream.

The scanned beam that results from the scanners 38a, 38b is then directed through a lens L1 to another mirror M2 that in turn reflects the radiation towards another beam splitter 40, which directs the scanned beam through a lens L2 and a quarter-wave plate 42 onto a portion of a sample 44, such as a retina, so as to illuminate a plurality of retinal vessels.

In many embodiments, an aiming device 50 can be used to facilitate alignment of the radiation onto a selected portion of the retina. A precise determination of a measurement location can allow obtaining repeated measurements from the same location over a selected time period, thereby enhancing measurement accuracy in temporal studies. More specifically, the aiming device 50 generates illumination light that is directed via a lens L3 to a mirror M3, which in turn directs the radiation along a path toward the beam splitter 40 that is collinear with the path of radiation from the source 32. The radiation from the aiming device 50 can then pass through the beam splitter 40 to be focused by the lens L2 through the quarter-wave plate 42 onto the retina. Hence, by appropriate positioning of the patient's head such that the aiming device is targeting a desired retinal portion, it can be ensured that the interrogating radiation is incident on the same retinal portion.

The scanning of the interrogating radiation from the source 32 over the retina causes the illumination of a plurality of retinal vessels through which the labeled cells are flowing. As noted above, upon excitation by this illuminating radiation, the labeled cells, and more particularly their fluorescent labels, emit fluorescence radiation. At least a portion of this fluorescence radiation, which is typically red shifted relative to the interrogating radiation, exits the eye and is reflected by the beam splitter 40 to the mirror M2, which in turn directs the fluorescence radiation to the lens L1. The lens L1 in turn converges the fluorescence radiation towards the scanning mirrors 38a, 38b. Since the fluorescence radiation is generated in response to the scanned interrogating radiation, it exhibits a similar scanning pattern (e.g., a circular pattern) as that of the interrogating radiation. The passage of the fluorescence radiation in a reverse direction through the scanner 38a, 38b, however, undoes the scanning and hence results in a fluorescence radiation beam that is stationary in a plane perpendicular to its propagation direction. This fluorescence radiation beam passes through the beam splitter or dichroic filter 36 to reach a color filter 46. The filter 46 allows the passage of the fluorescence radiation but substantially blocks radiation at shorter wavelengths. By way of example, the filter 46 can be a long-pass filter or a band-pass filter.

A lens L4 then converges the fluorescence radiation through a confocal pinhole 48 that is configured for the confocal detection of the fluorescence radiation. The pinhole 48 allows for the detection of fluorescence radiation emitted from a selected excitation volume, for example, the area of the retinal blood vessels, while minimizing detection of interfering photons that originate from regions beyond this volume. For example, even if such interfering photons reach the detection plane, they will not be generally in focus in that plane. In other words, the confocal arrangement substantially eliminates detection of radiation from out-of-focus fluorescent and/or scattering sources.

A detector, which is placed directly behind a pinhole 48, detects the emitted fluorescence radiation, and transmits the detected signals to an analysis module, such as a computer on which software for analysis of the data in accordance with the teachings of the invention is stored.

In this exemplary embodiment, the fluorescence detector is a photomultiplier tube 52 (PMT) that can be connected to a data acquisition card in a computer, that samples the received fluorescence radiation at a rate of about 100 kHz to generate digitized fluorescence signals for transmission to the analysis module. In other embodiments, the detector can be an avalanche photodiode (APD) or any other suitable detector known to those having ordinary skill in the art.

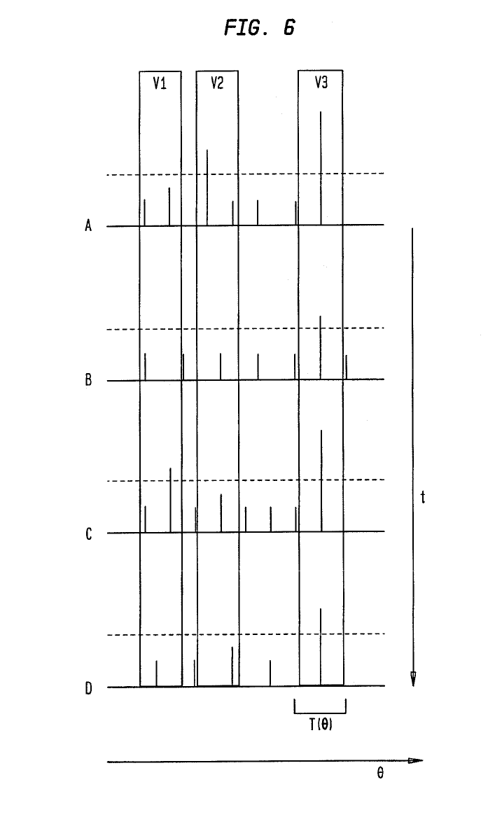

The analysis module can be configured to analyze the data in a variety of ways, as discussed further. In many embodiments, the circulating radiation beam scans the retina at a sufficiently fast rate so as to illuminate each of a plurality of retinal blood vessels multiple times during the time it takes for a labeled cell to traverse a region of a blood vessel corresponding to the illumination spot size. Hence, in such cases multiple fluorescence signals can be elicited from a single excited labeled cell. In some cases the fluorescent signals detected over a time interval (e.g., a time interval corresponding to four complete scans of the retina by the illumination beam) are examined to determine whether they include signals from labeled cell(s). FIG. 6 depicts fluorescence signals over four consecutive retinal scans (A, B, C, and D) in three blood vessels (V1, V2, and V3). By way of illustration, the temporal period corresponding to illumination of a particular blood vessel (V3) is depicted as T(θ) in each scan. For each scan A-D and for each vessel V1-V3, the fluorescence data collected during the temporal period T is examined to determine whether it contains a signal from a cell, e.g., by considering whether it contains a signal with an amplitude above a predefined threshold. Such a threshold can be, for example, a multiple (e.g., twice) of the root-mean-square (rms) noise in the scan. For example, in this case, the threshold is indicated by a dashed line in each scan. Hence, during the temporal period T(θ), scans A, B, C, and D show fluorescence signals above the threshold. Therefore, the fluorescence signals in V3 are considered as emanating from a cell because they are above the threshold multiple consecutive times (four times in this case). In contrast, one fluorescence signal in V2 during scan A and another in V1 during scan C are considered noise although they are above the threshold because they are isolated and thus cannot be considered a cell (no other scan exhibits a fluorescence signal above the threshold from the blood vessel). Moreover, in some cases, in addition to comparing the amplitude of a signal with a predefined threshold, the temporal width of a fluorescent signal is compared with a predefined width to determine whether it should considered as a signal emitted by a labeled cell. In some embodiments, if a given number of scans show signals above the threshold over corresponding to illumination of a retinal vessel, the analysis module indicates the detection of a labeled cell that has traveled through the illuminated retinal blood vessel. A similar analysis can be performed with respect to fluorescence signals corresponding to other retinal vessels.

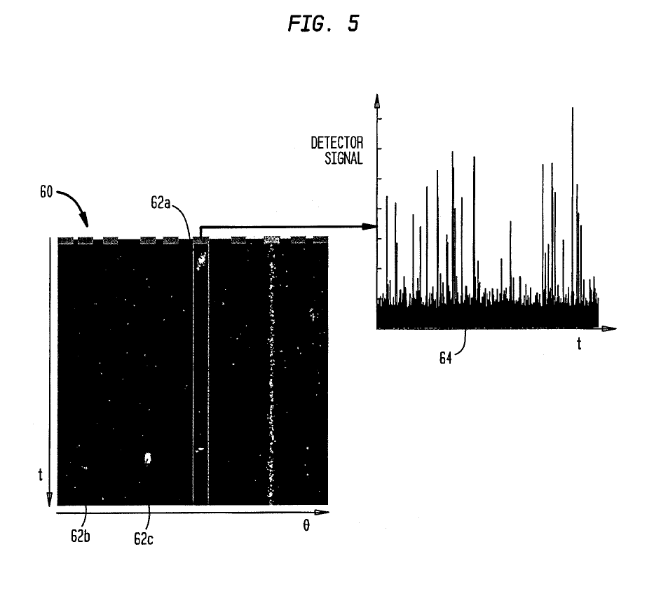

By way of further illustration, a retinal flow cytometer frame 60 shown in FIG. 5 (corresponding to FIG. 3B) illustrates a plurality of regions of interests 62a, 62b, and 62c, with each one representing the location of a retinal blood vessel (only fluorescently-tagged cells are visible in this representation). As the radiation beam is circularly scanned around the retina, as described above in FIG. 2, a graph 64 is created, as shown in FIG. 5, that represents the received fluorescence signals from the blood vessel obtained as a result of scanning. For each point on the graph 64 representing one region of interest, the fluorescence signal can be examined, e.g., in a manner discussed above, to determine whether it represents the presence of a cell, or is some other signal, for example, a signal representing background noise that can be disregarded in the cell count. For example, the graph 64 was obtained from blood vessel 62a over the course of one minute and contains 31 signals above a threshold and thus 31 cells are counted. A graph similar to the graph 64 is obtained for each region of interest, such as the regions of interest, 62b and 62c. The total cell count can be the sum of the cell counts from all the regions of interest.

As noted above, the fluorescence signals corresponding to a plurality of scans are examined in order to increase the reliability of the detection labeled cells and hence that of the cell count. In one exemplary embodiment, the number of scans examined can be based on speed of the scanned beam. For example, the scans corresponding to a maximum temporal interval during which a cell of interest would remain within a region of interest (an illuminated region) of a retinal blood vessel can be employed. The received signal for each of those scans is compared at the location of each region on interest with a predefined signal threshold, as shown in FIG. 6 and described above, and if a threshold number of those scans signals representing the presence of a labeled cell, then it can be concluded that the received signal is a legitimate signal representing a labeled cell and can be included in the cell count.

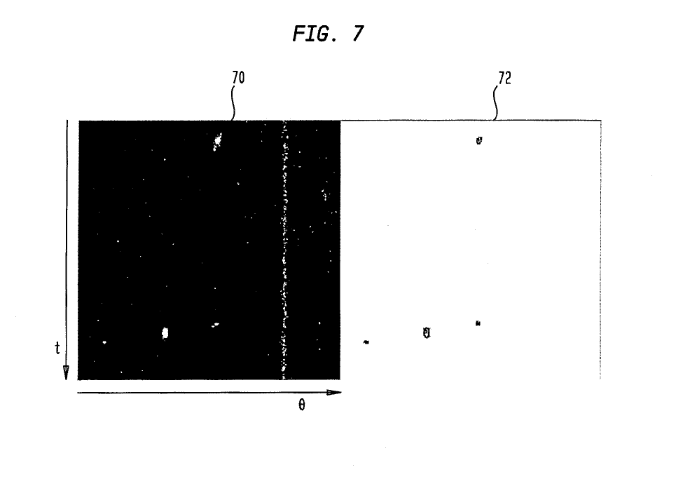

In another exemplary embodiment, the analysis of the fluorescence signal obtained progresses over a multitude of whole scans by analyzing the fluorescence signal frame by frame. Each still frame can be viewed as a matrix X pixels wide (mapping the angular position of the scanning spot and thus the size of a feature) and Y pixels high (representing progressing time), where each pixel contains a number that represents the amplitude of the detector and, thus, the brightness of the fluorescence (recall FIG. 3B). Therefore, each still frame can be viewed as a two-dimensional fluorescence signal (FIG. 7, image 70). The threshold previously described can be applied to this fluorescence signal to convert it to a binary data set. That is, those pixels that are below the threshold are set to zero (or OFF), and pixels that are above the predefined threshold are set to one (or ON). Thus, low level background noise is eliminated. The remaining signal contains individual ON pixels that appear due to noise and groups of adjacent ON pixels that represent cells. The analysis continues to enumerate the area that is occupied by pixels that are ON. It is known that a signal arising from a cell must be at least a predetermined number of pixels wide (xᵢ), due to the physical size of the cell (in the current embodiment, a cell occupies at least five pixels in x direction). It is furthermore known that a cell must be a predefined number of pixels high (yⱼ), due to the time it takes the cells to pass the excitation site (in the current embodiment, a cell is detected at least four times, thus must occupy at least four pixels in y direction). Therefore, the analysis software considers signals as arising from a cell when a group of ON pixels is at least xᵢ times yⱼ square pixels large. All smaller groups are eliminated and only the remaining groups of pixels are counted as cells (FIG. 7, image 72).

In the above embodiment, a spot of interrogating radiation scans over a retinal portion (e.g., a circular retinal portion) in a continuous fashion, thus illuminating not only a plurality of retinal vessels that support a significant blood flow but also other retinal portions that lack such vessels. These other retinal portions typically do not provide substantial contributions to the emitted fluorescence radiation, but can be a source of noise in the detection process. In some embodiments, the scanning radiation is selectively activated (or more generally modulated) so as to illuminate a plurality of retinal vessels but have a vanishing (or more generally a low intensity) over the retinal portions lying between those vessels. In this manner, fluorescence signals from the labeled cells flowing the illuminated vessels can be elicited while reducing noise caused by the interaction of the illuminating radiation with other retinal portions and minimizing thermal load on the retina due to the laser radiation.

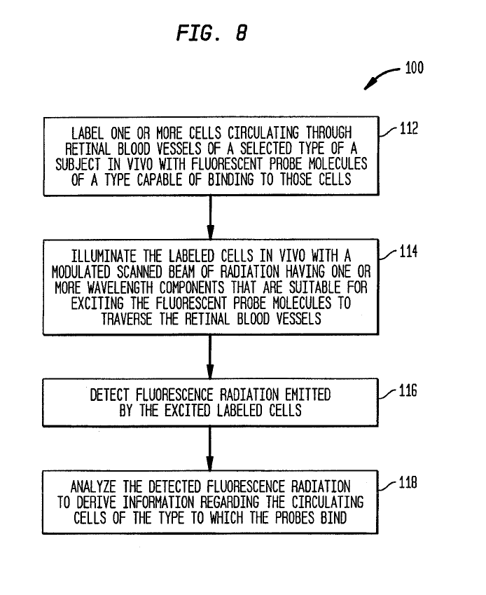

By way of illustration, FIG. 8 presents a flow chart depicting various steps of another exemplary embodiment of a method according to the teachings of the invention for performing retinal flow cytometry in which in an initial step 112, similar to step 12 in FIG. 1, one or more cells of a selected type circulating through the vasculature of a subject, e.g., a patient, are labeled in vivo with fluorescent probe molecules of a type capable of binding to those cells. In step 114, one or more retinal blood vessels of the subject are illuminated in vivo with a modulated scanned beam of radiation having one or more wavelength components that are suitable for exciting the fluorescent probes. A “modulated scanned beam,” as used herein, refers to a beam of radiation that is scanned over tissue (e.g., retinal tissue in this case) while its intensity is varied (modulated). Such modulation of the beam's intensity can be achieved, e.g., by periodically activating and deactivating the beam. Alternatively, the modulation can be achieved by varying the beam's intensity without deactivating the beam, or a combination of varying the intensity and at times deactivating the beam. For example, in some embodiments, the scanned beam can be modulated by controlling a radiation source to illuminate retinal blood vessels only when the beam intersects with a blood vessel of interest. This can be advantageous as it allows collecting fluorescence signals only when the beam is intersecting with the vessels, which can decrease the amount of noise collected and can streamline the analysis of the collected data. Similar to steps 16 and 18 in FIG. 1, in step 116, the fluorescence radiation elicited from the labeled cells by the illuminating radiation can be detected, and in step 118, the detected fluorescence can be analyzed so as to derive information regarding the circulating cells of the type to which the probes bind.

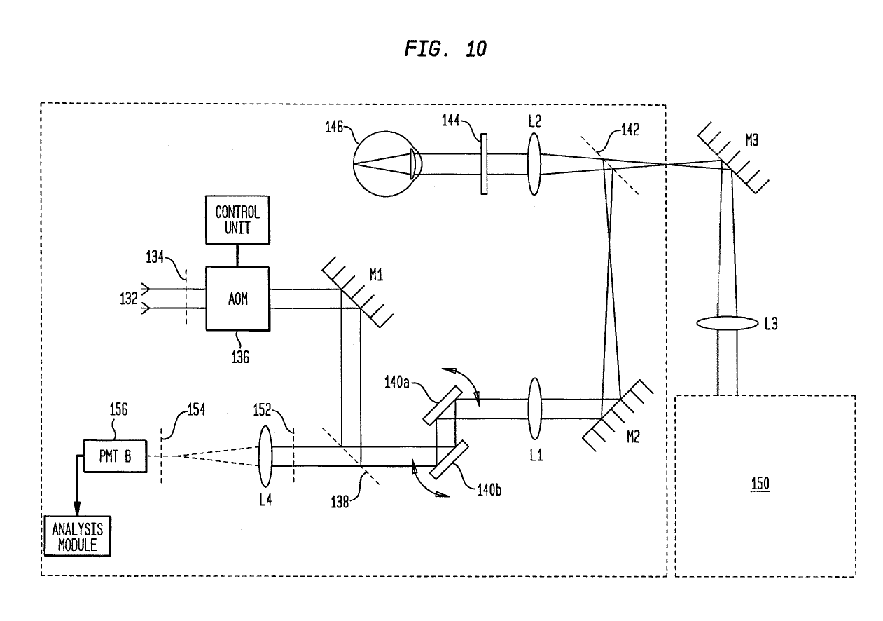

FIG. 10 schematically illustrates a system 130 for performing retinal flow cytometry according to another embodiment suitable for performing the aforementioned method of flow cytometry delineated in the flow chart 100 of FIG. 8. The system 130 includes a radiation source 132 that is capable of generating radiation having one or more wavelengths suitable for exciting labeled cells circulating through a subject's retinal vessels. An acousto-optic modulator (AOM) 136 receives radiation from the source 132 after its passage through a neutral density filter 134. As known in the art, the AOM 136 can modulate the intensity of the incoming beam via acoustic waves (e.g., at a frequency of tens of MHz) in a medium through which the incoming beam propagates. Such interaction can diffract in a time-varying manner some of the input beam into a new direction. Thus, the output beam of the AOM 136 (e.g., the beam corresponding to zeroth order diffraction) can exhibit an intensity modulation. The depth of modulation can be adjusted via the amplitude of the acoustic wave (e.g., in some cases, the beam intensity can periodically be reduced to vanishing values).

The modulated beam is then reflected by a mirror M1 to a beam splitter 138, which in turn directs the modulated beam to a scanner composed of a pair of scanning mirrors 140a, 140b, which similar to the system 30 discussed above, swivel about two orthogonal directions relative to one another to cause the beam to scan according to a desired pattern (e.g., along a circular path). The scanned beam is then directed via a convergent lens L1 to a mirror M2, which in turn directs the beam to a beam splitter 142. The beam splitter 142 directs the scanned beam to a convergent lens that focuses the beam onto the retina 146 though a lens L2 and a quarter-wave plate 144 onto the retina 146.

Similar to the system 30 described previously, the system 130 can include an aiming device 150 that can allow aligning the scanned beam onto a particular retinal portion. The aiming device 150 can provide an illuminating beam (e.g., visible radiation) that can be directed onto the retina via a lens L3, a mirror M3, though the beam splitter 142 to the lens L2, which in turn focuses the illuminating radiation through the waveguide 144 onto the retina 146. As discussed above, the co-linearity of the path of this illuminating radiation and the radiation from the source 132 allows positioning a radiation spot from the source 132 onto a selected retinal portion. In some cases, the aiming of the beam can be done after the scanning mirrors 140a, 140b are turned on, for example, using a manual procedure in which an operator visually places the beam on the retina as imaged by the aiming device 150. In other cases, the aiming of the beam can be done before the scanning mirror 140a, 140b are turned on. For example, the aiming device 150 can be configured to automatically determine the locations of blood vessels, and the portions of the retina to be illuminated can be determined prior to turning on the source 132.

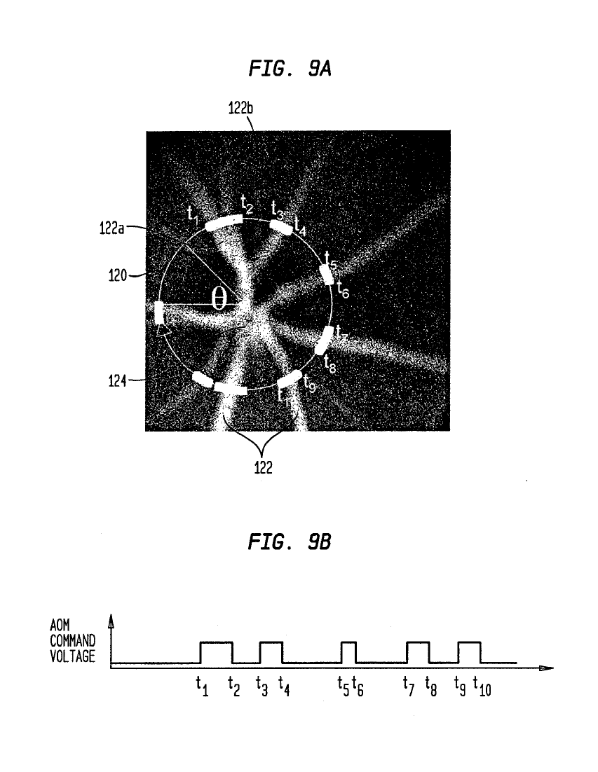

A control unit can apply control voltages to the AOM 136 such that the scanned radiation beam would be modulated so as to have a peak intensity as it scans over a retinal vessel and have a substantially lower intensity (e.g., zero intensity) as it moves between those vessels. By way of illustration and as discussed further below, such control (command) voltages are shown schematically in FIG. 9B in connection with a radiation beam illuminating a plurality of retinal blood vessels (FIG. 9A) during time intervals corresponding to those control voltages.

The modulated scanned radiation can excite the fluorescent labels of cells circulating through the illuminated retinal blood vessels. In response to the excitation, the fluorescent labels emit radiation that leaves the eye and is directed via the waveguide 144, the lens L2, and the beam splitter 142 to the mirror M2. The mirror M2 in turn directs the returning fluorescence radiation via the lens L1 to the scanning mirrors 140a, 140b. As discussed in detail above, the passage of the returning fluorescence radiation through the scanner can undo the scanning of the fluorescent beam to generate a stationary beam (a beam not showing substantial movement in a plane perpendicular to its propagation direction). The fluorescent beam then passes through the beam splitter 138 to be focused by the lens L4 via the color filter 152 through a confocal pinhole 154 onto a detector 156 (in this case a photomultiplier tube).

Similar to the previous embodiment, an analysis module receives the detected fluorescence signals and analyze those signals, e.g., in a manner discussed above, to obtain information regarding one or more circulating cell types of interest.

In some cases, the detection system is gated in synchrony with the modulation of the radiation beam from the source 132 such that the detection system is exposed to radiation returning from the eye only during those time intervals in which the radiation from the source illuminates the retinal vessels. For example, in this case, the control unit can apply control signals to the detector 156, and/or an adjustable aperture (not shown) placed in front of the detector 156, in synchrony with command voltage signals applied to the AOM 136 to activate the detector only during those time intervals in which one or more retinal blood vessels are illuminated.

FIG. 9A is an exemplary illustration of a fluorescence image in retinal blood vessels visualized with cells labeled with fluorescent probe molecules using a modulated scanned beam of radiation. The scanned beam of radiation 120 is illustrated as it is scanned around retinal blood vessels 122 extending radially from an optic nerve head 124. The beam 120 is modulated to illuminate the vessels 122 only when the beam 120 is intersecting with a vessel 122. For example, the beam 120 is activated at time t1 as it approaches the location of a vessel 122a so as to illuminate that vessel upon reaching it. The beam continues to illuminate the vessel 122a until time t2 when it has completely traversed the vessel 122a. During the temporal interval between t2 and t3, the beam 120 is scanning an area of the retina in which there are no major blood vessels, so the beam 120 is deactivated. The beam 120 is again activated at time t3 when the beam 120 begins to intersect another vessel 122b. This modulation of the beam 120 continues as the beam 120 is scanned around the retina. FIG. 9B illustrates a graph showing AOM command voltage versus time, indicating the modulation of the scanned beam of radiation as it scans the retinal blood vessels shown in FIG. 9A. When the beam 120 is intersecting retinal blood vessels 122, the control voltage jumps to some voltage above zero, activating the AOM (or alternatively the radiation source) to illuminate the vessels 122. When the beam 120 is not intersecting a vessel 122, the control voltage is substantially zero to deactivate the beam 120.

In another embodiment, the excitation beam can be split such that two circular paths are scanned over the retina around a common center (e.g., a smaller inner ring and a larger outer ring). In addition to various analyses described above, in such an embodiment, the velocity of the cells passing through the double-circle illumination pattern can be measured. For example, a cell passing through the two illumination circles will generate one fluorescence signal when passing one illumination circle and another fluorescence signal when passing the other illumination circle (that is, the scanned beam following the inner circle will elicit one fluorescence signal from such a cell and the scanned beam following the outer circle will elicit another fluorescence signal for that cell). Dividing the known separation (distance) between the inner and the outer circles by the time delay of the two fluorescence signals will yield the velocity of the cell.

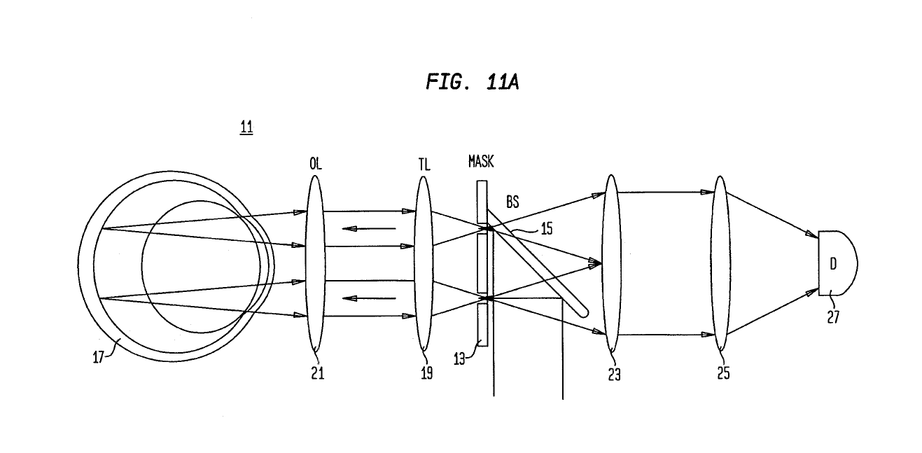

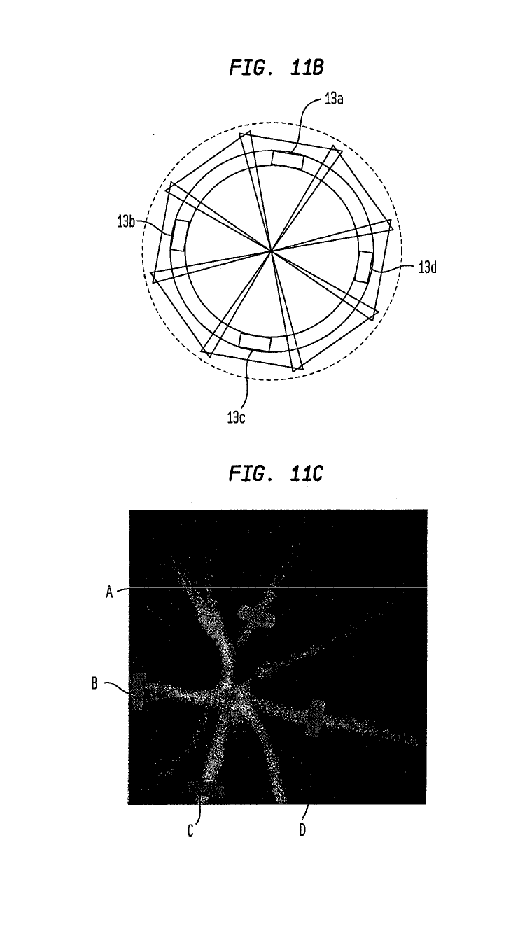

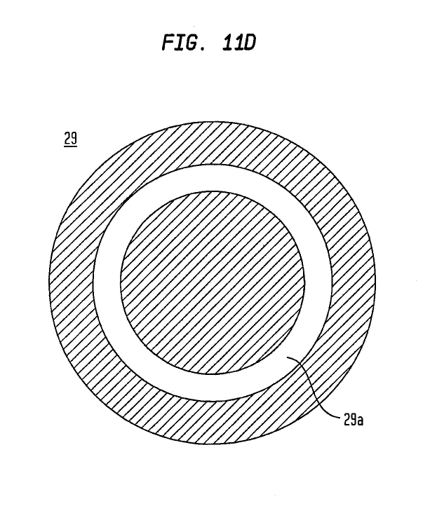

In some other embodiments, rather than scanning a radiation beam over the retina, a stationary beam together with a mask are employed to illuminate one or more retinal blood vessels. By way of example, in one implementation of such an embodiment, a mask having multiple apertures can receive radiation from a source and project the radiation through the apertures onto portions of a plurality of retinal blood vessels so as to excite fluorescent-labeled cells flowing through those vessels. With reference to FIGS. 11A and 11B, an apparatus 11 according to such an embodiment for performing retinal flow cytometry can include a mask 13, which receives radiation from a source (not shown) via reflection from a beam splitter 15. The mask 13 includes a plurality of rectangular apertures 13a, 13b, 13c and 13d (herein collectively referred to as apertures or slits 13). The mask allows the passage of the radiation through its apertures (rectangular slits in this exemplary implementation) and blocks the remainder of the incident radiation. The radiation passing through each slit is projected onto the retina 17 by a tube lens 19 and an objective lens 21.

Each slit can be aligned with a portion of a retinal blood vessel such that the light projected through that slit onto the retina can excite fluorescent-labeled cells flowing through that vessel. In some cases, each slit can be aligned such that the slit intersects perpendicularly with a respective blood vessel. By way of further illustration, FIG. 11C shows the projections of the slits 13 onto the retina in the form of four rectangular-shaped illumination areas (A, B, C, and D), each corresponding to a retinal blood vessel

The fluorescence radiation emitted by the excited labeled cells is directed via the lenses 21 and 19 onto the slits and passes through the slits and the beam splitter to be focused by lenses 23 and 25 onto a detector 27. In some embodiments, the detector 27 is a detector array of one detector element per slit. The detected fluorescence can be analyzed in a manner discussed above to count the cells from which fluorescence radiation is detected and to obtain information regarding those cells. By way of example, apparatus 11 can include an analysis module (not shown) coupled to the detector that is configured to analyze the detected fluorescent signals.

In this implementation, the rectangular apertures 13 are embedded in adjustable paddles that can rotate around the center of the mask. Hence, the apertures can be rotated to be aligned with different retinal blood vessels. In an alternative embodiment, the mask can include a ring-shaped aperture through which a complete illumination circle can be projected onto the retina. Such a mask is shown schematically in FIG. 11D, which include a ring-like aperture 29a. Alternatively, the aperture can extend about a portion of a ring (e.g., it can be in the form of a half of a circle). Those having ordinary skill in the art will appreciate that other aperture shapes and types can also be utilized

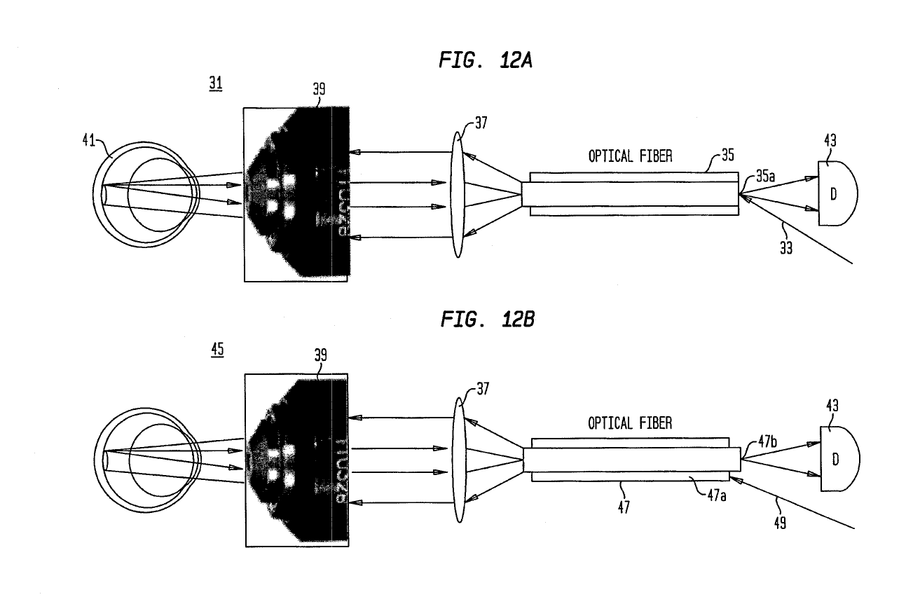

In another embodiment, an illumination (excitation) circle can be generated by forcing an optical waveguide to emit a donut-shaped mode of emission, which can be projected onto the retina to provide a circular excitation pattern. By way of example, FIG. 12A schematically depicts such a system 31 in which a radiation beam 33 generated by a source (not shown) is coupled into the core 35a of an optical fiber 35 at an angle such that the light output from the fiber core would exhibit a donut-shaped mode. The donut-shaped light output at the fiber tip is imaged by lenses 37 and 39 onto a subject's retina 41 so as to excite fluorescent-labeled cells flowing through one or more retinal blood vessels. The fluorescence radiation emitted by the excited cells is coupled via the lenses into the fiber 35, which in turn transmits the returning fluorescence radiation onto a detector 43. Though not shown, in some cases one or more optical components, such as lenses, disposed between the optical fiber 35 and the detector 43 can focus the returning fluorescence radiation onto the detector. The detected fluorescence radiation can then be analyzed, e.g., by an analysis module (not shown), in a manner discussed above to count the cells from which fluorescence is detected and to obtain information regarding those cells.

FIG. 12B schematically depicts an apparatus 45 according to another embodiment for performing flow cytometry, which similar to the previous embodiment utilizes an optical fiber 47 to generate a radiation beam having a donut-shaped mode for illuminating the retina. In this embodiment, however, a radiation beam 49 generated by a source (not shown) is coupled into the cladding 47a of the optical fiber. The radiation then travels through the cladding and leaves the fiber in the form of a ring of radiation. This radiation can then be imaged by the lenses 37 and 39 onto a circular portion of the retina 41 to intersect a plurality of retinal blood vessels and to excite one or more fluorescent-labeled cells flowing through those vessels. The fluorescence radiation emitted by the excited cells is then coupled by the lenses into the core 47b of the fiber 47. The fluorescence radiation leaves the fiber to be incident on the detector 43. As noted in connection with the previous embodiment, in some cases one or more lenses (not shown) can focus the fluorescence radiation leaving the fiber onto the detector. The detected fluorescence radiation can be analyzed, e.g., by an analysis module (not shown), to count the cells from which fluorescence radiation is detected and to obtain information regarding those cells.

To further illustrate various aspects of the invention, the following example is provided to illustrate the use of the systems of the invention discussed above to monitor labeled cells in vivo. It should, however, be understood that the example is not intended to necessarily indicate the optimal results (e.g., optimal cell counts) that can be achieved by employing the devices discussed above.

### Example 1

In one exemplary embodiment (FIG. 4), the retinal flow cytometer is essentially a confocal line-scanning microscope. It was assembled as a front end for a confocal microscope that serves as an aiming device to verify the plane and region probed by the retinal flow cytometer. In the retinal flow cytometer, two phase-locked resonant galvanometer scanners (for example, available from Thorlabs) circularly steer the beam of the excitation laser (635 nm, Radius, Coherent) at a rate of 4.8 kHz. The pupil formed by the scanner is projected telecentrically into the shared pupil at the entrance aperture of a 20× infinity-corrected microscope objective (NA 0.42, M Plan APO). The excitation beam of the retinal flow cytometer is not expanded, thus underfilling the objective's aperture, yielding a measured spot diameter (1/e²) of 13 μM in air and a depth of focus of 320 μm (i.e., twice the Raleigh range). Excited fluorescence is descanned by the resonant scanning minors and detected through a dichroic long-pass filter at 45° and through a 670 nm bandpass filter. Out-of-focus signal is rejected by a 400 μm pinhole in front of the photomultiplier tube (PMT) (for example, R3896, available from Hamamatsu).

A photomultiplier tube (PMT) signal is fed into a variable scan analog frame grabber (for example, Snapper 24, Active Silicon). Each circular scan is displayed as a straight horizontal line of 500 pixels in length; consecutive scans are oriented as adjacent lines. Consequently, the line frequency along the negative y axis of the resulting 500×500 pixel image equals the sampling rate in each blood vessel. Furthermore, retinal blood vessels that diverge outward from the optic nerve head (ONH) appear as straight vertical structures, as the x axis maps the angular position of the flying spot, for example, shown in FIG. 3 described above. Streaming raw data was recorded with imaging software developed in house in Mac OS X for postprocessing.

For initial feasibility experiments about 10⁶ DiD-labeled lymphocytes (freshly isolated from extracted lymph nodes) were injected into an anesthetized BALB/c mouse. DiD (Vybrant DiD, Molecular Probes/Invitrogen) is a lipophilic dye used as a membrane marker with an emission maximum at 670 nm after excitation with a 635 nm laser. Injected cells were counted with the retinal flow cytometer both by placing the circular scan around the ONH as well as over a single retinal blood vessel. For comparison, cell count was also enumerated in the ear of the same mouse with an in vivo flow cytometer (IVFC) using slit excitation. To determine the cell count, several data sets were recorded in each location. The cell count is presented as the mean and standard deviation among the data sets from the same experiments (Table 1).

TABLE 1

| | Retinal Flow Cytometer in Retinal Vessels | | | | | | |
|---|---|---|---|---|---|---|---|
| | | | ROI and Perl | | ImageJ Particle | | IVFC |
| | Manual Count | | Code | | Analysis | | MatLab |
| | ONH | Single | ONH | Single | ONH | Single | Single |
| Cells/min | 269 | 31 | 238 | 25 | 224 | 26 | 53 |
| St-Dev | 39 | 3 | 69 | 4 | 61 | 3 | 11 |

Cell counts from the retinal flow cytometer were determined by multiple independent observers manually inspecting each frame of the recorded files. To explore automated counting techniques, the cells were also enumerated using two different software algorithms and the results were compared with the manual counts. For software analysis, two basic criteria need to be satisfied for a signal to be counted as a single cell: the fluorescence signal (1) needs to be distinguishable from background signal in amplitude and (2) needs to have a minimum temporal width. Assuming a maximum cell velocity of 10 mm/s, a circular scan of about 5 kHz should intercept a lymphocyte of about 8 μm in size at least four times. Consequently, signals shorter in time than four pixels were not counted as cells, independent of their amplitude. In both software analyses, blood vessel locations were identified by the cells passing through the movie frames in vertical lines. Regions outside major vessels were excluded from the analysis, since no valuable information can be expected.

In one software approach we determined the cell count by particle analysis in ImageJ. After noise reduction using ImageJ's mean filter (an isolated bright pixel cannot constitute a cell and is replaced with the mean of its surrounding 3×3 matrix), the original 8-bit data set was converted to a binary movie; the same settings for the threshold were used in the analysis of all experimental data sets. In ImageJ's particle analysis function, the size of contacts to be counted was specified according to our temporal width criterion. Thus, ImageJ particle analysis counted and outlined structures that were identified as cells (FIG. 7).

In a second software counting approach, we extracted a plot of pixel intensity over time for each of the probed vessels using a movie stitching utility that was developed in house in Mac OS X. A region of interest (ROI) was placed in the position of each blood vessel. The ROI is equivalent to the slit in IVFC; the frequency of the circular scan is similar to the sampling rate in IVFC. Within the ROI, a minimum filter was applied for noise reduction; isolated bright pixels cannot constitute a cell and are replaced with zero. By integrating along the horizontal axis within the ROI and dividing by the number of pixels spanned by the ROI, a normalized pixel value was computed. The signal spikes in the resulting time trace were counted using code written in Perl that identifies cells based on the height and width of their fluorescence signal (FIG. 5).

The results demonstrate that counting fluorescently labeled cells in circulation is feasible with the retinal flow cytometer. Probing the blood vessels that diverge from the ONH resulted in a cell count that was five times higher than that derived from the IVFC in the ear of the same mouse (Table 1). Thus, it can be inferred that the retinal flow cytometer probes a sample volume that is five times larger than that of the IVFC, although 10 blood vessels are probed. The diameter of a typical retinal blood vessel in a 30-day-old mouse is about 25 μm, while blood vessels of about 35 μm are targeted in IVFC. Consequently, cell counts in a single ear vessel are expected to be about twice as high as counts in a single retinal vessel; we measured an IVFC versus single retinal vessel count ratio of 1.7 (Table 1).

Retinal cell counts evaluated by software were about 15% lower than manual counts, as the height threshold was set to a fairly high level in order to avoid miscounting noise as cells and thus increasing specificity. The specificity of the ImageJ results (Specificity=1−incorrectly counted/total manual count) was 95%, determined by comparing the marked cells in the analyzed movie to cells in the raw movie. The high specificity of the software ensured that an individual cell in the raw file was correctly identified as a single cell by software.

In alternate exemplary embodiments of the invention, the retinal flow cytometers as described above can have various features. For example, a retinal flow cytometer can have a higher numerical aperture. Smaller focal diameter and shorter depth of focus can result in improved sensitivity and increased signal-to-noise ratio, by increasing irradiance for excitation and refining depth sectioning. However, the smallest spot size in the retina is limited by the numerical aperture (about 0.2-0.3) as well as by aberrations of the mouse eye that may prevent achieving the diffraction limit.

One of ordinary skill in the art will appreciate further features and advantages of the invention based on the above-described embodiments. Accordingly, the invention is not to be limited by what has been particularly shown and described, except as indicated by the appended claims. Those having ordinary skill in the art will appreciate that various changes can be made to the above embodiments without departing from the scope of the invention. All publications and references cited herein are expressly incorporated herein by reference in their entirety.

What is claimed is:

1-20. (canceled)

21. A method of performing flow cytometry, comprising:
labeling one or more cells of a selected type of a subject with one or more fluorescent probe molecules while the cells circulate in the subject;
scanning an excitation radiation beam over a selected area of the subject to excite the one or more fluorescent probe molecules; and
detecting fluorescence radiation emitted by the one or more fluorescent probe molecules in response to the excitation radiation.

22. The method of claim 21, further comprising analyzing the detected fluorescence radiation so as to derive information regarding the circulating cells of the selected type.

23. The method of claim 22, wherein the analyzing step comprises determining whether a signal-to-noise ratio of a detected fluorescence signal exceeds a pre-defined threshold.

24. The method of claim 23, wherein the analyzing step further comprises identifying a plurality of detected fluorescence signals collected from a vessel over a time period shorter than a time interval required for passage of a labeled cell through an illuminated portion of the vessel as corresponding to a cell count if a pre-defined number of the signals exhibit an intensity exceeding the threshold.

25. The method of claim 21, wherein a rate of the scan is such that one or more cells flowing through a vessel are illuminated at least once as the beam is scanned.

26. The method of claim 22, wherein the derived information provides a cell count of the plurality of cells relative to a corresponding count measured previously.

27. The method of claim 26, wherein the relative cell count can be indicative of progress of a treatment protocol applied to the subject.

28. The method of claim 22, wherein the derived information provides an absolute cell count of the plurality of cells.

29. The method of claim 28, wherein the absolute cell count can be indicative of any of presence of a disease and progress of a treatment protocol.

30-47. (canceled)
